(19) United States
(12) Patent Application Publication
(10) Pub. No.: US 2006/0150279 A1
(43) Pub. Date: Jul. 6, 2006

(54) Bioremediation with transgenic plants

(76) Inventors: Alexander Pavlovich Sorokin, Norwich (GB); Galina Genadievna Brychkova, Beer-Sheva (IL); Nikolai Alexandrovich Kartel, Minsk (BY); Jonathan Dallas George Jones, Norwich (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/536,923

(22) PCT Filed: Dec. 5, 2003
(86) PCT No.: PCT/GB03/05322

§ 371 Date: Oct. 27, 2005

(30) Foreign Application Priority Data
Dec. 5, 2002 (GB) 0228444.6

(51) Int. Cl. A01H 1/00; C12N 15/82; C02F 3/32

(52) U.S. Cl. 800/284; 210/602

(57) ABSTRACT

Methods are provided for phytoremediating environments contaminated with at least one heavy metal or oil hydrocarbon (or both), the methods comprising use of transgenic plants which express one or more enzymes having rhamnosyltransferase activity (e.g. encoding the rh1A and rh1B genes derived from Pseudomonas aeruginosa). Also provided are related methods, materials and processes for producing and using such transgenic plants.

### TECHNICAL FIELD

The present invention relates generally to transgenic plants which are tolerant to metals and\or hydrocarbons, and related methods and materials, which useful for bioremediation.

### BACKGROUND ART

Nowadays, mankind faces a lot of environmental risks. Some are caused by the outcome of human activities, others by natural factors. The growth of the world population strengthens the need for food, fuel, fertilizers, and chemicals. In the future man will have to improve agriculture, industry and transport as well as produce more energy, but these activities are, at present, often associated with an increase in the heavy metals and oil hydrocarbons contamination of the environment. Such contamination, in its turn, may adversely influence the yield ratio and the quality of crops. Additionally, excessive concentrations of heavy metals can lead to serious diseases in humans as well as in animals¹. Because of its high toxicity, mutagenicity and carcinogenicity, oil hydrocarbons are likewise detrimental for all living organisms².

This problem has led to the development of numerous approaches to the treatment of contaminated sites³, some of which are based on natural or “green” technologies, for example using plants. These natural methods of decontamination are highly promising in combating the pollution caused by organic and inorganic substances⁴. Such plant-based group of remediation methods, known as phytoremediation, has proven its cost-effectiveness, efficiency; compatibility with other techniques, and environmental friendliness^{2,5}.

The improvements to plants offered by genetic engineering can in principle provide new possibilities for the phytoremediation of contaminated sites^{8, 19}. However the precise effects of such engineering in respect of particular substrates, and plant growth more generally (particularly where genes of prokaryotic origin are employed) cannot be easily predicted.

Thus it can be seen that the provision of transgenic plants with demonstrated effectiveness in the bioremediation of metals and\or hydrocarbons would provide a contribution to the art.

### DISCLOSURE OF THE INVENTION

The present inventors have created transgenic plants incorporating prokaryote genes which contribute to the biosynthesis of rhamnolipids. Rhamnolipids are formed from one or two molecules of rhamnose linked to one or two molecules of beta-hydroxydecanoic acid, and belong to the group of rhamnose-containing glycolipid biosurfactants¹¹ first described in 1949^{12, 14-15}.

In embodiments described below, transgenic Arabidopsis thaliana and Nicotiana tabacum plants have been prepared using rh1A and rh1B genes from Pseudomonas aeruginosa PAO1. Plants demonstrated tolerance to both oil hydrocarbons and copper contaminations in soil. They also promoted quick reclamation of soil by enhancing biodegradation of crude oil, in particular, the most stable C₁₂-C₁₈ hydrocarbon fractions. This is believed to be the first demonstration of any plant coping with co-contamination of soil with two different pollution agents: crude oil and copper. Since many polluted sites are co-contaminated with several agents, transgenic plants of the present invention can provide an effective instrument for their phytoremediation.

The rh1A and rh1B genes encoding rhamnosyltransferase from Pseudomonas aeruginosa, and their role in the biosynthesis of rhamnolipids, were already known in the art^{9, 10, 13} (see FIG. 6). The rh1A and rh1B proteins have been suggested to exist as a protein complex. It has been suggested that Rh1B is the catalytic protein of the rhamnosyltransferase, while the Rh1A protein is involved in the synthesis or transport of rhamnosyltransferase precursor substrates or perhaps in the stabilization of the Rh1B protein in the cytoplasmic membrane¹⁰. Jain et al. (1992, J. Ind. Microbiol. 10, 87-93) noticed that the addition of rhamnolipid biosurfactant from P. aeruginosa to a hydrocarbon-soil mixture (tetradecane, hexadecane and pristine) enhanced degradation of all hydrocarbons within two months. However, the addition of the bacterial cells alone to the mixture did not significantly increase biodegradation. In other work, rhamnolipid biosurfactants were analysed for their ability to remove hydrocarbons from sandy-loam and silt-loam soils, and gravel (Van Dyke et al. Can. J. Microbiol. 39, 1071-1078 (1993); Scheibenbogen et al. J. Chem. Techn. Biotech. 59, 53-59 (1994); Harvey et al. Bio/Techn. 8, 228-230 (1990)).

However transgenic rhamnolipid expression has not previously been demonstrated as the basis for effective phytoremediation plants. Several attempts have been made to engineer plants resistant to specific hydrocarbons (French et al. “Biodegradation of explosives by transgenic plants expressing pentaerythritol tetranitrate reductase”. Nat. Biotech. 17, 491-494 (1999); Doty et al. “Enhanced metabolism of halogenated hydrocarbons in transgenic plants containing mammalian cytochrome P450 2E1”. Proc. Natl. Acad. Sci. USA 97, 6287-6291 (2000)). However, no successful technology has been previously developed for bioremediation of oil hydrocarbon contaminated soils.

In the examples below, the plants grow and produce seeds in soils with copper content exceeding 1 g per 1 kg of wet soil. The plants with the rh1A gene and plants with both genes acted as perfect metal excluders. Unlike the control plants, the plants with the rh1B gene and-those with the rh1A gene demonstrated an ability to enhance degradation of large amounts of crude oil and minimal accumulation of oil hydrocarbons.

Thus in one aspect the invention provides a method of phytoremediating an environment which is contaminated with at least one heavy metal or oil hydrocarbon, which method comprises:

(a) providing a transgenic plant, which plant expresses at least one heterologous nucleic acid encoding an enzyme having rhamnosyltransferase activity,

(b) planting or locating said transgenic plant in said environment.

The term “phytoremediation” is used herein as it will be well understood by those skilled in the art to include any of (i) phytoextraction, when metal-accumulating plants are employed to extract metals from contaminated soil⁶; (ii) phytostabilization, when metal-tolerant plants, (known as “excluders”), are used to reduce or exclude the environmental risk in heavy metals⁴; (iii) phytodegradation, when plants are applied to degrade nonvolatile hydrocarbons, thus removing them from the environment⁷. As shown in the examples, the present invention is a realistic strategy for phytoremediation of oil and metal co-contaminated soil. In preferred embodiments of the invention the phytoremediating process is either one or both of phytostabilizing heavy metal contaminants or phytodegrading oil hydrocarbons.

The “environment” may be any site which it is desired to phytoremediate e.g. which is contaminated with organic or metal pollutants. Examples include sites contaminated with oil near drilling rigs, oil refineries, near mines, power stations, metalurgical plants and so forth.

Some aspects and embodiments of the invention will now be discussed in more detail.

Heavy Metals

The “heavy metals” to which the invention may be applied may include any one or more of lead, copper, cadmium, nickel, mercury, arsenic, selenium strontium or zinc.

Stability constants for the rhamnolipid complexation with various metals were found to be similar or higher than conditional stability constants for metal binding with other organic compounds of soil and follows the order (from strongest to weakest) Al³⁺>Cu²⁺>Ph²⁺>Cd²⁺>Zn²⁺>Fe³⁺>Hg²⁺>Ca²⁺>Co²⁺>Ni²⁺Mn²⁺>Mg²⁺>K⁺ (Ochoa-Loza et al. J. Environ. Quality 30, 479-485 (2001).

The level of heavy metal in the environment may depend on the metal itself. For example it may contain between 50 mg/kg and 15 000 mg/kg of lead or 3000 mg/kg of copper; 0.2 mg/kg and 12000 mg/kg of nickel; 1.2 mg/kg and 1,500 mg/kg of cadmium and so on. Although legislation varies, it may be preferred to use the present invention at sites in which metal concentrations exceed locally permitted levels e.g. lead 0-500 mg/kg, for copper 0-100 mg/kg, for nickel 0-20 mg/kg, for cadmium 0-1 mg/kg.

Preferably metal accumulation coefficient (C_{MA}) of the plants used in the present invention (C_{MA}=[Cₛ]/[Cᵣ], where Cₛ is the copper concentration in shoot, Cᵣ is the relevant copper concentration in the rhizosphere) is less than 20% of that of non-transgenic plants, where the heavy metal is copper present at 1000 mg/kg. As can be seen in the examples below, in preferred embodiments (rh1A gene in Arabidopsis) the C_{MA} is less than 5% of the controls (0.05 vs. 1.3).

As described below, in preferred embodiments transgenic plants of the invention can live in the soil with a 300-fold higher Cu concentration than wild-type plants. However, those skilled in the art will appreciate that even those with a lesser improvement, say 100-fold, or 200-fold, will still have utility in phytoremediation.

Oil Hydrocarbons

The “oil hydrocarbon” to which the invention may be applied may be any of those which may pollute the environment (see, for example, References 1 and 18) including substituted hydrocarbons and derivatives. Particular pollutants may include petrolium-derived hydrocarbons e.g. crude oil. Other target pollutants include organogalogens (Benzene, 1,2,-dichloro-, benzene, pentachloro), linear or cyclic aliphatic hydrocarbons, aromatic hydrocarbons. Although legislation varies, it may be preferred to use the present invention at sites in which hydrocarbon concentrations exceed locally permitted levels e.g. around 50 mg/kg.

Rhamnosyltransferases

In the plants of the invention, the nucleic acid encoding the or each rhamnosyltransferases are “heterologous” to the plant. The term “heterologous” is used broadly in this aspect to indicate that the nucleic acid, gene or sequence of nucleotides in question have been introduced into said cells of the plant or an ancestor thereof, using genetic engineering, i.e. by human intervention. A heterologous gene may in principle replace an endogenous equivalent gene, but will generally be additional to the endogenous genes of the genome i.e. is non-naturally occurring in cells of the plant type, variety or species.

Preferred rhamnosyltransferases will be those involved in the synthesis of monorhamnolipids, as these are believe to play a role in both heavy metal tolerance and in enhancing of oil degradation. dirhamnolipids are believed to give benefits primarily in respect of low oil hydrocarbon accumulation.

Preferably, the gene encoding the enzyme having rhamnosyltransferase activity will be rh1A or rh1B from a procaryote. These encode rhamnosyltranserase and participate in the biosynthesis of rhamnolipids¹³. As shown in the Examples, even when acting alone these genes provide advantageous properties, a result which is quite unexpected in view of the prior art (see e.g. Ref 10, or Rahim R, Ochsner U A, Olvera C, Graninger M, Messner P, Lam J S, Soberon-Chavez, G (2001) Cloning and functional characterization of the Pseudomonas aeroginosa rh1C gene that encodes rhamnosyltransferase 2, an enzyme responsible for dirhamnolipid biosynthesis. Molecular Microbiology, 40: 708-718). The results of the inventors suggest that, although both individual genes produce both mono- and dirhamnolipids, rh1A favours the former while rh1B favours the latter. Plants overexpressing both genes produce significant quantities of both mono- and dirhamnolipids.

Preferably the rh1A or rh1B gene are derived from Pseudomonas aeruginosa. Optionally a gene encoding rh1C may be included also.

Preferably the plant comprises nucleic acids encoding both of these genes, although as described in the examples, even the single genes provide advantageous properties. Optionally it may further comprise other heterologous nucleic acids encoding other genes involved in the biosynthesis of rhammolipids. P Where reference is made to the rh1A or rh1B genes, it should be understood that, except where the context demands otherwise, variants, both natural and artificial, may be used as long as the variant forms retain the ability to encode a polypeptide with an appropriate corresponding enzymatic(rhamnosyltransferase) capability.

A “variant” nucleic acid molecule as used herein will encode a functional polypeptide (e.g. which is a variant of the polypeptides encoded by the rh1A or rh1B genes, and which may cross-react with an antibody raised to said polypeptide). Variants may be used to alter the phytoremediating properties characteristics of plants as described above.

Generally speaking variants may be naturally occurring nucleic acids, or they may be artificial nucleic acids. Variants may include homologues of the rh1A or rh1B genes. Other variants may be modified e.g. with respect to GC\AT ratios, in order to improve expression in plants (which modification may, but preferably will not, lead to any change in the encoded enzyme). Particularly included are variants which include only a distinctive part or fragment (however produced) corresponding to a portion of the relevant gene, encoding at least functional parts of the polypeptide. Also included are nucleic acids corresponding to those above, but which have been extended at the 3′ or 5′ terminus. The term “variant” nucleic acid as used herein encompasses all of these possibilities. Variants will be substantially homologous the wild type genes. Homology may be at the nucleotide sequence and/or encoded amino acid sequence level. Preferably, the nucleic acid and/or amino acid sequence shares at least about 60%, or 70%, or 80% homology, most preferably at least about 90%, 95%, 96%, 97%, 98% or 99% similarity or identity. Such similarity or identity may be as defined and determined using FASTA and FASTP (see Pearson & Lipman, 1988. Methods in Enzymology 183: 63-98). Parameters are preferably set, using the default matrix, as follows:

Gapopen (penalty for the first residue in a gap): −12 for proteins/−16 for DNA

Gapext (penalty for additional residues in a gap): −2 for proteins/−4 for DNA

KTUP word length: 2 for proteins/6 for DNA.

The homology between nucleic acid sequences may be determined with reference to the ability of the nucleic acid sequences to hybridise to each other. One common formula for calculating the stringency conditions required to achieve hybridization between nucleic acid molecules of a specified sequence homology is (Sambrook et al., 1989): Tₘ=81.5° C.+16.6 Log [Na+]+0.41 (% G+C)−0.63 (% formamide)−600/#bp in duplex

Plants

The invention, in another aspect, provides a transgenic plant which plant comprises at least one heterologous nucleic acid encoding an enzyme having rhamnosyltransferase activity as described herein. Such plants are adapted for use in the phytoremediation methods discussed above. Plants of the invention may be produced by regeneration from a transformed plant cell as discussed below. In addition to the regenerated plant, the present invention embraces all of the following: a clone of such a plant, seed, selfed or hybrid progeny and descendants (e.g. F1 and F2 descendants). The invention also provides a plant propagule from such plants, that is any part which may be used in reproduction or propagation, sexual or asexual, including cuttings, seed and so on. It also provides any part of these plants, which in all cases include a plant cell expressing a heterologous rhamnosyltransferase enzyme.

For the avoidance of any doubt, the propagules of such plants (seeds, cuttings, and so on) may be used in the methods of phytoremediation discussed herein, and the term “plant” should be construed accordingly, unless context demands otherwise.

Although exemplified herein by N. tabacum and A. thaliana, the plants of the invention may be any appropriate to the environment and climate, including trees.

Nucleic Acid Constructs

The invention further provides a plant construct or vector comprising a nucleotide sequence an enzyme having rhamnosyltransferase activity. Such vectors are adapted for producing phytoremediating plants above.

More specifically, in a further aspect of the invention, there is provided a recombinant nucleic acid vector suitable for transformation of a plant, which vector comprises a nucleotide sequence encoding on either or both of the rh1A or rh1B genes from Pseudomonas aeruginosa (or variants thereof as discussed above).

“Vector” is defined to include, inter alia, any plasmid, cosmid, phage or Agrobacterium binary vector in double or single stranded linear or circular form which may or may not be self transmissible or mobilizable, and which can transform a plant cell.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148), the teaching of which is herein incorporated by reference. Suitable vectors may include plant viral-derived vectors (see e.g. EP-A-194809).

The (or each) nucleotide sequence encoding the enzyme having rhamnosyltransferase activity will preferably be under the control of, and operably linked to, a promoter or other regulatory elements for transcription in a host plant cell. By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

The promoter may be an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK, the teaching of which is herein incorporated by reference. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, that is to say those that may be used to confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Methods and Processes

Thus according to one aspect of the present invention there is provided a method of producing a phytoremediating plant, or a method of manipulating, and preferably improving the phytoremediating properties of a plant, comprising introducing into a plant cell a vector as described above. The method preferably entails causing or allowing recombination between the vector and the plant cell genome to introduce at least nucleotide sequence encoding the (or each) rhamnosyltransferase (e.g. the rh1A or rh1B gene are derived from Pseudomonas aeruginosa) into the plant genome. It may optionally further comprise the steps of regenerating the plant and cultivating it.

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711 -87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species.

There has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233). The skilled person will appreciate that the particular choice of a transformation technology may be determined by its efficiency to transform certain plant species depending on the ease of use as well as the experience, preference and skill of the person practising the invention.

In a further aspect of the invention, there is disclosed a plant host cell containing a heterologous construct according to the present invention, as described above.

The host cell (e.g. plant cell) is preferably transformed by the construct, that is to say that the construct becomes established within the cell, altering one or more of the cell's characteristics and hence phenotype e.g. with respect to rhamnolipid biosynthesis. The alteration in the phytoremediating properties may be assessed by comparison with a plant in which the nucleic acid has not been so introduced (see e.g. Examples and FIG. 1).

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

All publications, patent applications, and references to sequences cited in this specification are herein incorporated by reference as if each individual publication, patent application and sequence were specifically and individually indicated to be incorporated by reference.

This includes reference to the sequences of:
The rh1A gene from Pseudomonas aeruginosa
The rh1B gene from Pseudomonas aeruginosa

which have Accession number L28170 in NCBI. See also Chayabutra, C., and Ju, L.-K. 2000. Degradation of n-hexadecane and its metabolites by Pseudomonas aeruginosa under microaerobic denitrifying conditions. Appl Environ Microbiol 66:493-498; Ochsner, U. A., Fiechter, A., and Reiser, J. 1994. Isolation, characterization, and expression in Escherichia coli of the Pseudomonas aeruginosa rh1AB genes encoding a rhamnosiltransferase involved in rhamnolipid biosurfactant synthesis. J Biol Chem 269:19787-19795).

The invention will now be further described with reference to the following non-limiting Figures and Examples. Other embodiments of the invention will occur to those skilled in the art in the light of these.

### FIGURES

FIG. 1. Rhamnolipid content of different plants analyzed by the Thin Layer Chromatography method (TLC)¹⁶. The bands 1-6 correspond to:

1-rhamnolipids from Ps.aeruginosa PAO1

2-rhamnolipids from the root zone of the sterile cultivated plants with both genes (rh1A+rh1B)

3-rhamnolipids from the root zone of the sterile cultivated plants with rh1B gene (as can-be-seen, they produce both monorhamnolipid and dirhamnolipid).

4-rhamnolipids from the root zone of the sterile cultivated plants with rh1A gene (as can be seen, they produce only monorhamnolipid).

5-control plants (there were no bands in the nontransgenic control and control plants with empty vector)

6-rhamnolipids from Ps.aeruginosa PAO1

FIG. 2. Copper accumulation demonstrated by Arabidopsis plants grown in contaminated soil (1020 mg of Cu per 1 kg of wet soil). For comparison, plants grown in uncontaminated soil demonstrated the following levels of Cu accumulation: control plants—9.47 mg/kg; plants with empty vector—8.63 mg/kg; plants with rh1A—18.07 mg/kg; plants with rh1B—8.44 mg/kg; plants with rh1A and rh1B—8.73 mg/kg.

FIG. 3. (A) Tobacco plants in the soil with the 912 mg kg⁻¹ wet soil Cu amount. A nontransgenic control plant (left), a transgenic plant with rh1A gene (right). (B) Copper accumulation demonstrated by tobacco plants grown in contaminated soil. For comparison, plants grown in uncontaminated soil (3 mg kg⁻¹ wet soil) demonstrated the following levels of copper accumulation: control plants—42 mg/kg; plants with rh1A—39 mg/kg.

FIG. 4. (A) Oil hydrocarbon accumulation demonstrated by plants grown in the soil contaminated with 2.4% of crude oil. 1—a nontransgenic control plant; 2—a control plant with empty vector; 3—a transgenic plant with rh1A gene; 4—a transgenic plant with rh1B gene; 5—a transgenic plant with rh1A+rh1B genes.

FIG. 5. Degradation of crude oil demonstrated by plants grown in contaminated soil. (Top) The 1.3% level of contamination. (Bottom) The 2.4% level of contamination.

FIG. 6. The rhamnolipid biosynthesis pathway in Pseudomonas aeruginosa includes the rh1A, rh1B and rh1C genes encoding two rhamnosyltransferases involved in transfer of one or two rhamnose molecules to β-hydroxydecanoyl-β-hydroxydecanoate.

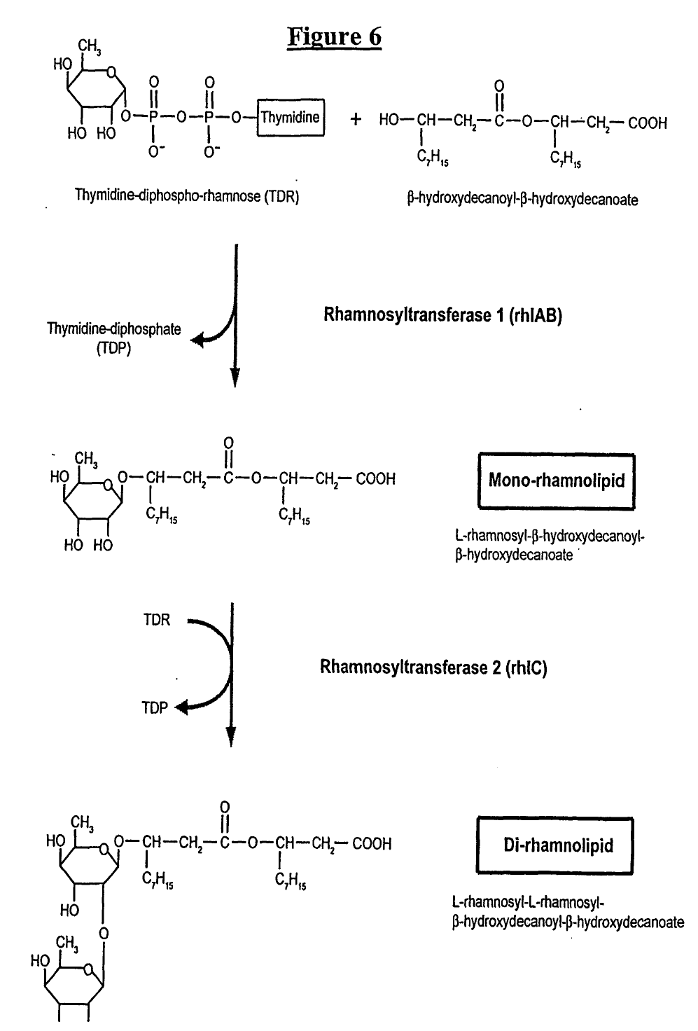

FIG. 7. Degradation of specific hydrocarbons by tobacco rh1A transgenic plants. Degradation of oil fraction #4 (Mozir Refinery Factory, Belarus) containing C₆-C₂₀ hydrocarbons was analysed.

### EXAMPLES

### Example 1

### Creation of Transgenic Plants for Remediation Purposes

Using cloned rh1A and rh1B genes from Pseudomonas aeruginosa, we created specific constructs for constitutive expression in plant tissues. We have originated the plants with the three types of constructs: first, with rh1A gene; second, with rh1B gene; and the third, with both genes, (a rh1A+rh1B construct).

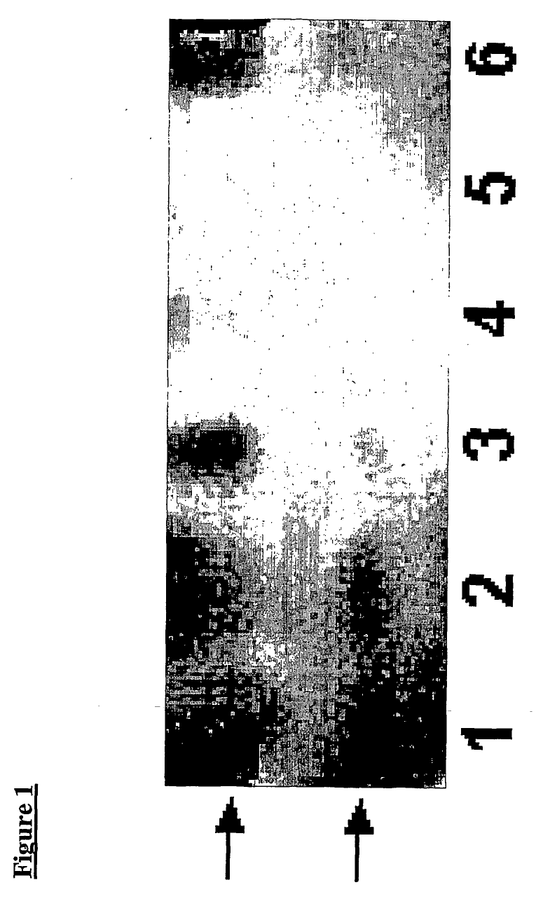

Subsequently, we produced 30 independent lines of transgenic Nicotiana tabacum, (10 lines per each construct), and 30 independent lines of transgenic Arabidopsis thaliana, (10 lines per each construct), in order to study the effect of rhamnolipid overexpression in planta on metal resistance and oil degradation. To serve as a control for our experiments, we created plants with empty vector. The presence of the transgenes was tested by the method of Southern hybridization. The rhamnolipid content was analyzed by the Thin Layer Chromatography method (TLC)¹⁶ (FIG. 1).

The areas of TLC plates containing bands of rhamnolipids were scraped and MS analysis was carried out on these samples for control and rh1A transgenic plants. Two major peaks with masses of 503.3 and 649.3 corresponding to the mono-rhamnolipid and di-rhamnolipid were observed for the rhamnolipid surfactant JBR-425. Peaks of the same masses were also identified in rh1A transgenic, but not in control plants (results not shown). Therefore transgenic lines produced both mono-rhamnolipid and di-rhamnolipid and could consequently be used at contaminated sites as a donor of these compounds.

### Example 2

### Transgenic Plants vs. Copper Contamination

An increase in heavy metal concentration in soil is proven to have visible effects on plants: it shortens shoots, yellows leaves and, eventually, kills the plant¹⁷. The level of the above toxic effect depends on the type of heavy metal and its bioavailability as well as on the plant.

After preliminary tests with heavy metals we decided to use copper to exemplify heavy metal contamination. The basic plant culture for our tests was Arabidopsis thaliana. Our research program comprised the following experimental objects: a) uncontaminated control soil; b) contaminated soil; c) transgenic plants with rh1A gene; d) transgenic plants with rh1B gene; e) transgenic plants with rh1A and rh1B genes; f) control transgenic plants with empty vector; g) normal nontransgenic control plants.

Objects from c to g were tested both in uncontaminated and contaminated soils, readings taken from the measurements of both their root zones and shoots. We applied a concentration of 1067 mg Cu per 1 kg of wet soil, which is considered toxic¹⁷. Our aim was to study the levels of Cu concentration by measuring its content in rhizosphere and in shoots respectively. We took measurements at the beginning and in the end of the 45-day period of the plant growth. The experiments were done with the help of a flame atomic absorption spectrophotometer.

Our research demonstrated that injection of 1067 mg Cu into 1 kg of wet soil (atomic absorption analysis detected only as much as 1020 mg of available Cu in 1 kg of wet soil), killed both nontransgenic control Arabidopsis plants and control Arabidopsis plants with empty vector.

For transgenic Arabidopsis with the three types of constructs (rh1A, rh1B, rh1A+rh1B) the results were different. Transgenic Arabidopsis plants with rh1A gene and transgenic Arabidopsis plants with both genes could grow on the contaminated soil without exhibiting any toxic symptoms. Not only did they give seeds but also demonstrated great biomass production in the soil contaminated with copper. As for plants with rh1B gene, their growth in contaminated soil was suppressed. However, we observed no chlorosis (Table 1).

TABLE 1
Comparative morphological analysis of Arabidopsis
plants grown in soil contaminated with copper.

| | Shoot length, | | Dry plant | | Seed | |
|---|---|---|---|---|---|---|
| | cm | | biomass, g | | production | |
| | | in | | in | | in |
| | in | contam- | in | contam- | in | contam- |
| | control | inated | control | inated | control | inated |
| | soil | soil | soil | soil | soil | soil |
| Control | 12 | Died | 0.047 | 0.003 | + | − |
| plants | | | | | | |
| Plants | 15 | Died | 0.03 | 0.002 | + | − |
| with empty | | | | | | |
| vector | | | | | | |
| Plants | 11 | 24 | 0.023 | 0.106 | + | + |
| with rhlA | | | | | | |
| Plants | 18 | 4 | 0.045 | 0.031 | + | +/− |
| with rhlB | | | | | | |
| Plants | 17 | 35 | 0.055 | 0.515 | + | + |
| with rhlA | | | | | | |
| and rhlB | | | | | | |

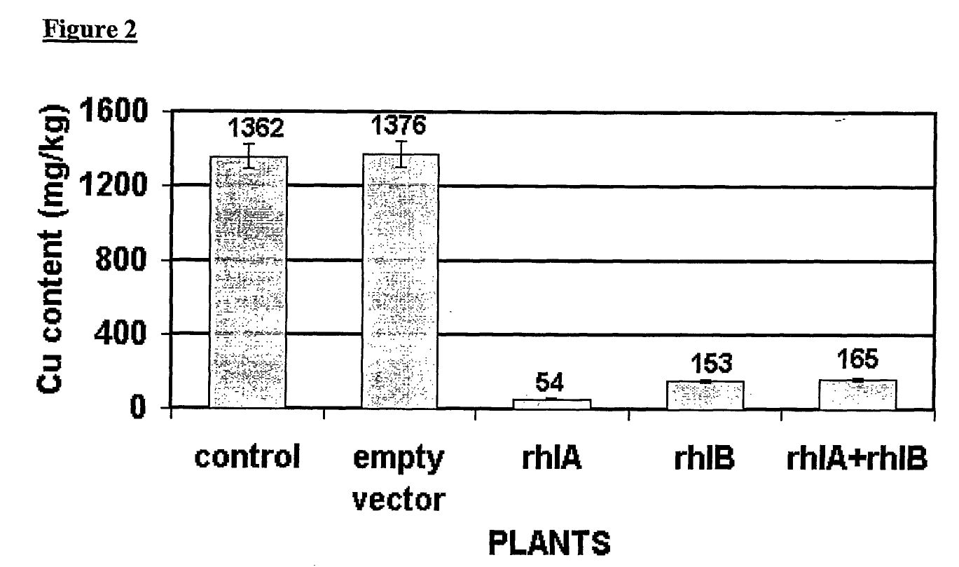

The atomic absorption analysis helped determine the levels of copper accumulation measuring Cu content in all plants grown both in uncontaminated control soil and in contaminated soil (FIG. 2).

The level of copper accumulation in plants was evaluated with the help of the metal accumulation coefficient (C_{MA}) which we derived through the following equation:
C_{MA}=[Cₛ][Cᵣ],

where Cₛ is the copper concentration in shoot, Cᵣ is the relevant copper concentration in the rhizosphere.

In the uncontaminated control soil, the Cu accumulation coefficient (C_{MA}) was 3.5 in both transgenic and nontransgenic plants. In contaminated soil, C_{MA} was 1.3 for the control plants with empty vector and nontransgenic control plants. The plants with rh1B gene had C_{MA} equal to 0.15. Plants containing constructs with both rh1A and rh1B genes demonstrated C_{MA} at 0.16. In plants with rh1A gene, C_{MA} proved the best, less than 0.05. Evidently, in transgenic plants with the three types of constructs, the low figure of C_{MA} implies very small copper content in shoots in comparison with Cu content in the rhizosphere.

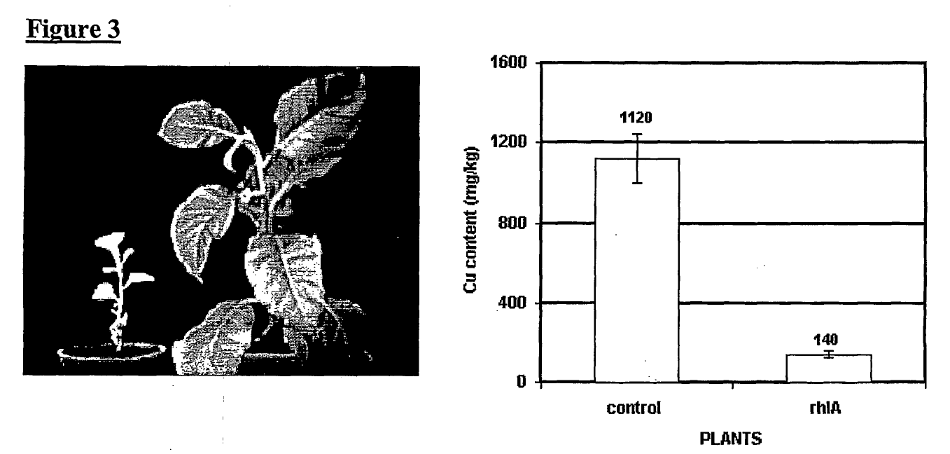

We also performed corresponding tests on Nicotiana tabacum. We verified control tobacco and rh1A-containing tobacco plants using a copper content which was lower than in our tests on Arabidopsis. We applied the copper concentration of 912 mg kg⁻¹ wet soil. Control tobacco plants survived in this copper concentration, but demonstrated slow growth, chlorosis of leaves and infertility. Conversely, transgenic tobacco plants with rh1A gene grew without any toxic symptoms and gave normal seeds (FIG. 3A). Atomic absorption analysis showed that these GM tobacco plants with rh1A gene were able to exclude copper from their above-ground tissues (C_{MA}=0.07) in contrast to the ordinary nontransgenic tobacco plants (C_{MA}=1.11) (FIG. 3B).

Generally, all transgenic plants with the three types of constructs could live in the soil with a 300-fold higher Cu concentration than the wild-type plants. We applied the concentration levels equivalent to, or even exceeding, those present in industrially contaminated sites. The most interesting and promising results were seen in plants with rh1A gene. These metal-tolerant Arabidopsis and tobacco plants can apparently grow in very contaminated soil by avoiding any metal transport from roots to shoots. Thus, they behave as true excluders. This suggests that introduction of the rh1A gene into plants allow them to exclude toxic heavy metals from their tissues and grow in extremely polluted soils. Plants with this gene may be used for phytostabilization on sites contaminated with heavy metals.

### Example 3

### Oil Hydrocarbon Accumulation by Plants

At the present time, oil hydrocarbons are among the most virulent organoxenobiotics. These pollutants can translocate from roots to the above-ground parts of plants¹. The toxic effect of oil hydrocarbons on plants ranges from the decrease in transpiration to plant mortality¹⁸.

The procedure of our experiments with oil was analogous to that applied with copper. We tested the same objects. We tested both Arabidopsis and tobacco. The contamination levels we applied were 1.3% and 2.4% of crude oil per wet soil.

Both nontransgenic control plants and transgenic plants with empty vector were visibly badly affected by oil. They showed infertility and a decrease in the biomass production. By contrast, transgenic plants with all the three types of constructs appeared to thrive on oil: they produced seeds and great biomass (Table 2).

TABLE 2
Comparative morphological analysis of Arabidopsis plants
grown in soil contaminated with 2.4% of crude oil.

| | Shoot length, | | Dry plant | | Seed | |
|---|---|---|---|---|---|---|
| | cm | | biomass, g | | production | |
| | | in | | in | | in |
| | in | contam- | in | contam- | in | contam- |
| | control | inated | control | inated | control | inated |
| | soil | soil | soil | soil | soil | soil |
| Control | 12 | 2 | 0.047 | 0.015 | + | − |
| plants | | | | | | |
| Plants | 15 | 3 | 0.03 | 0.015 | + | − |
| with empty | | | | | | |
| vector | | | | | | |
| Plants | 11 | 13 | 0.023 | 0.030 | + | + |
| with rhlA | | | | | | |
| Plants | 18 | 11 | 0.045 | 0.105 | + | + |
| with rhlB | | | | | | |
| Plants | 17 | 19 | 0.055 | 0.025 | + | + |
| with rhlA | | | | | | |
| and rhlB | | | | | | |

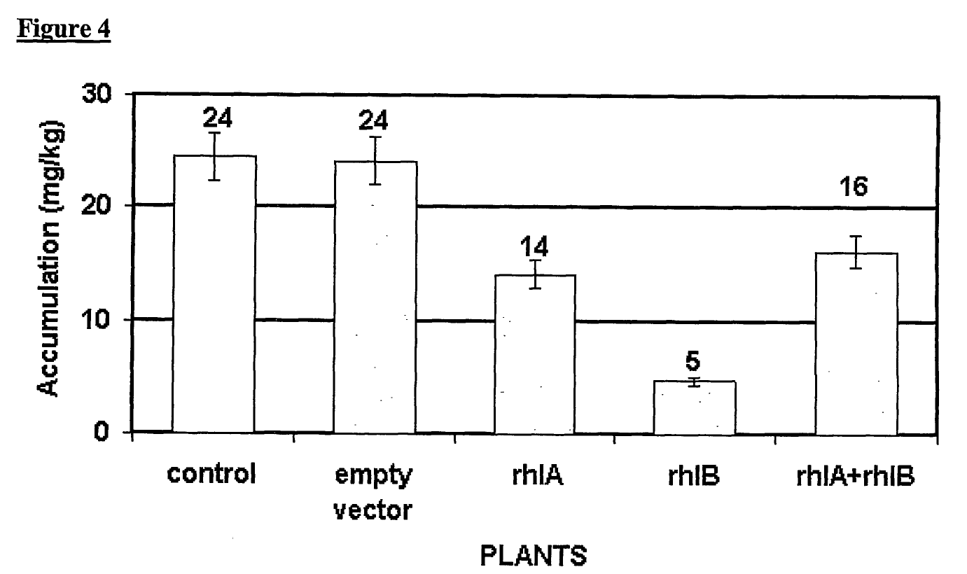

We then applied GC-FID (Gas Chromatography-Flame Ion Detection) method to test oil hydrocarbon presence in Arabidopsis plant tissues. Plant growth period in contaminated soil was 45 days. Nontransgenic control plants and transgenic plants with empty vector demonstrated the highest levels of oil hydrocarbon accumulation. All transgenic plants with our constructs exhibited low levels of oil hydrocarbon contents in their tissues after growth in soil polluted with crude oil. An intriguing fact is that plants with rh1B gene grown on the 2.4% crude oil level showed the highest biomass production and accumulated a very small amount of oil hydrocarbons (FIG. 4A).

Thus both Arabidopsis and tobacco transgenic plants were tolerant of 2% oil pollutant in the soil and could withstand concentrations up to 5% oil without visible developmental symptoms (not shown).

To investigate whether plant tolerance to crude oil required microbial interaction, tobacco leaf explants from control and rh1A transformed plants were transferred to sterile medium supplemented with 5 or 7.5% oil. As expected, explants from rh1A transgenic plants developed callus, while explants from control transgenic plants died after 1-2 weeks of cultivation.

### Example 4

### Biodegradation of Oil Hydrocarbons by Transgenic Plants

The next stage of our experiments was to determine possible biodegradation of oil hydrocarbons and, if so, to evaluate its level. Biodegradation can be defined as a basic natural process that helps remove nonvolatile hydrocarbons from the environment⁷. An increase in the bioavailability of hydrocarbons by rhamnolipids¹³ intensifies the degree of bioremediation of oil-contaminated soils.

Rates of oil hydrocarbon degradation were studied by measuring oil content in soil (more exactly, in the rhizosphere). The contamination levels we applied were 1.3% and 2.4% of crude oil per wet soil. Period of plant cultivation lasted 45 days. We compared the quantities of oil hydrocarbons in soil at the beginning and at the end of the 45-day period both for nontransgenic and transgenic plants with three types of constructs. The results showed that in the case of transgenic plants the amount of oil hydrocarbons in soil after this period dropped considerably.

The percentage of oil degradation can be estimated with the help of degradation coefficient (C_{d}) in the following equation:
C_{d}=(1−Aᵣ/Aᵢ)×100%,
where Aᵣ is the remaining amount of oil hydrocarbons in soil, Aᵢ is the initial amount of oil hydrocarbons in soil.

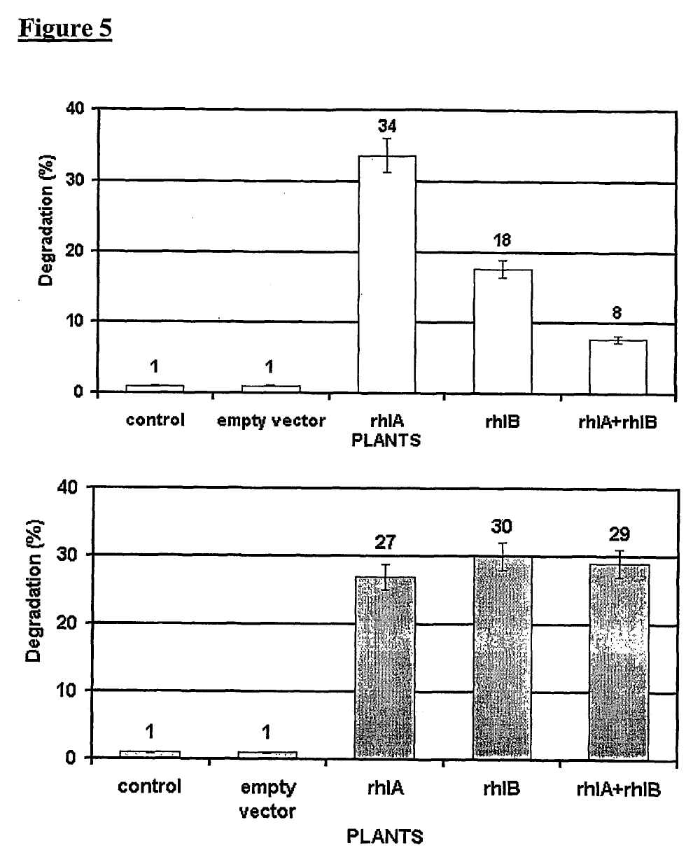

In the polluted soil (1.3% of oil) with nontransgenic control plants, the oil hydrocarbon concentration decrease was insignificant. This decrease is connected with the natural hydrocarbon degradation which occurs due to the influence of bacteria, sunlight, oxygen, and other factors. Control plants with empty vector showed similar results. In contrast to the control plants, rather high degradation coefficients were registered in plants with rh1B and rh1A+rh1B genes. However, the highest percentage of oil degradation in the root zone was demonstrated by the plants with rh1A gene (C_{d}=35%) (FIG. 5A).

With the 2.4% level of oil contamination, the picture was as follows. It is known that the presence of greater amounts of oil hydrocarbons in soil leads to a decrease in the rates of remediation processes. This was clearly seen in the case of plants with rh1A gene. For example, for rh1A construct in 1.3% soil, C_{d} was at 35%, while for 2.4% oil we had 25%. However, in case of rh1B construct we have a reverse process, (C_{d} increased from 18% to 25%) (FIG. 5B).

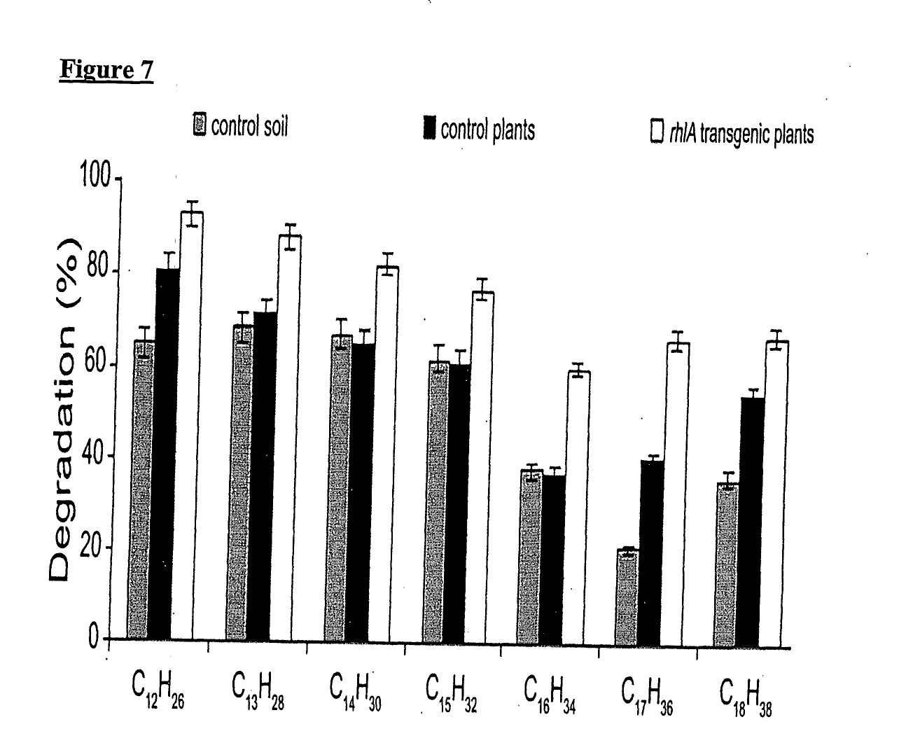

We also assessed degradation of specific hydrocarbons by the tobacco rh1A transgenic plants. Degradation of oil fraction #4 (Mozir Refinery Factory, Belarus) containing C₆-C₂₀ hydrocarbons was analysed. The final degradation was assessed after 119 days of plant growth on soil supplemented with 2% oil fraction # 4 per wet soil. Natural degradation of oil was observed both in soil without plants and in soil supporting control plants (FIG. 7). Degradation of hydrocarbons C₆-C₉ in contaminated soil was accomplished in 3-5 weeks, while a low rate of degradation was observed for C₁₂-C₁₆ fraction. There were no differences in the rate of degradation for C₁₂-C₁₆ between control soil and control transgenic plants, although degradation was increased for C₁₇ (heptadecane) and C₁₈ (octadecane) under control plants. Transgenic plants overexpressing the rh1A gene showed a considerable increase in the degradation rate for all fractions of C₁₂-C₁₈ hydrocarbons, in particular, the degradation of the most stable fractions C₁₆-C₁₈ was increased by 32-45%.

Thus transgenic plants containing rhamnolipid genes demonstrated a high speed of soil reclamation from hydrocarbon pollution. We observed good results in the plants containing rh1A gene and plants with rh1B gene likewise. This indicates that the introduction of rhamnolipids into plants is favorable both for plant survival and phytoremediation. Such transgenic plants offer great potential for cleaning up sites contaminated with different amounts of oil hydrocarbons.

### Example 5

### rh1A Transgenic Plants are Tolerant to Copper and Oil Co-Contaminated Soil

We also analysed the potential use of rh1A expressing plants for growth on soils co-contaminated with both crude oil and copper. In addition, the ability of commercially available rhamnolipid biosurfactant to rescue growth of control plants on co-contaminated soils was tested. Control and rh1A transgenic plants were grown on soil co-contaminated with 1% oil and 1700 mg copper per kg of wet soil without or with addition of rhamnolipid biosurfactant. Control plants grew poorly on the contaminated soil relative to transgenic plants (not shown). The addition of the biosurfactant to the soil had a detrimental effect on the control plants, but facilitated growth of rh1A transgenic plants.

The control and rh1A transgenic plants were planted side by side onto control non-contaminated soil and soil co-contaminated with copper and oil to test whether the presence of rh1A transgenic plants could rescue control plants. No differences in growth were observed for plants growing on control soil, but on soil with oil and copper the rh1A transgenic plants could not rescue the control plants.

### Example 6

### Soil Bioremediation by Control and Transgenic Plants

The plants were removed after growing in contaminated soil (1% oil and 1700 mg copper per kg of wet soil), and the soil was reused to plant wild type plants. We observed a reduction in growth rate and yellowing of leaves for the plants grown on the contaminated soil previously used to support control plants. In contrast, control plants grown on soil previously used for rh1A transgenic plants, showed up to 2-fold higher growth rate compared to plants grown on soil used to support control plants (not shown). This growth rate, however, was only about 70% of that of the control plants growing on non-contaminated soil.

Experimental Protocols

Vector Construction.

pAS50 construct (35S-rh1A-nos—pGreen0229): Rh1A (made available by Dr U.Ochsner, University of Colorado) was amplified using the following primers:

forward (ClaI):
5′- TTTATCGATTGGGAGGTGTGAAATGCGGCGCGA-3′
reverse (XbaI):
5′- TTTTCTAGATGTTCAGGCGTAGCCGATGGCCAT-3′

The obtained fragment was cloned into pGEM-T easy vector and sequenced using BigDye Terminator Cycle Sequencing Kit (PE Biosystems).

SLJ8271 vector (made available by Dr J.Jones, Sainsbury Laboratory. John Innes Centre, UK) containing uida gene under 35S promoter was digested with ClaI-XbaI to remove uida and was replaced by rh1A gene. The cassette containing 35S promoter, rh1A gene and nos terminator was cut with EcoRI-Hind III enzymes and was introduced into the pGreen 0229 vector.

pAS51 construct (SP-rh1B-ags—pGreen0229): the rh1B gene was cut out from pUO58-19 (from Dr U.Ochsner, University of Colorado) with BbsI, filled in and digested with ClaI. The fragment was ligated into pGEM-7Zf (Promega) vector digested with EcoRI (filled in) and ClaI enzymes. The rh1B gene was excised from pGEM-7Zf with XbaI-SacI enzymes and inserted in XbaI-SacI sites of pGreen-ags-terminator vector. A superpromoter (PMSP-1, made available by Prof. S.Gelvin, Purdue University, USA) was added into KpnI-EcoRI sites, resulting in pAS51 construct, containing rh1B gene under the superpromoter.

pAS52 construct (35S-rh1A-nos-SP-rh1B-ags-pGreenO229): A three way ligation was carried out with EcoRI-HindIII fragment from pAS50 and EcoRI-SacI fragment from pAS51 into pGreen0229 digested with HindIII-SacI enzyme, resulting in pAS52 construct.

Plant Material

Arabidopsis thaliana and tobacco (Nicotiana tabacum cv. Petit Havana SR1) were grown with supplemental lighting in Climatic Cabinet (WTB Binger Labortechnic GmbH) under 16-hour day length and 24°/18° C. day/night temperature.

Agrobacterium-mediated transformation of Arabidopsis thaliana.

Plant material was grown in containment glasshouse with the temperature of 24° C. and 20° C. for day and night correspondently. A colony of Agrobacterium with appropriate construct was inoculated into 5 ml of LB medium containing 50 mg/l of kanamycin and incubated at 28° C. overnight. 2 ml of overnight culture were inoculated into 200 ml of LB medium supplemented with 50 mg/l of kanamycin and 150 μM of acetosyringone.

The culture was incubated for further 7-9 hours at 28° C. and collected by centrifugation at 5000 rpm for 15 min. The pellet was resuspended in infiltration medium (½ MS medium, 5% sucrose, 500 μ/l Silwet L-77 (Union Cambridge, USA)). Arabidopsis flower buds were dipped into Agrobacterium culture for 60-90 sec and the plants were laid flat in propagator and covered by plastic roof to maintain humidity. The propagator was uncovered in 24-36 hours and the plants were moved to the normal growing conditions mentioned above.

Nicotiana Tabacum Plant Transformation.

The isolation and transformation of tobacco protoplast were done in accordance with Potricus²⁰. Protoplasts were isolated from the fully expanded leaves of tobacco plant in enzyme solution (18 hour/25° C./dark). Each millionth of protoplast suspension was transformed with 10 μg of corresponding sterile linearized plasmid DNA in PEG solution. For selection of stable transformed clones, protoplasts in solid 0.6% (w/v) K3 medium were cultivated in A medium containing, where appropriate, Km (50 mg L⁻¹) or PPT (20 mg L⁻¹) at 24° C./500 1×. The shoots, spontaneously outgrowing from selected colonies on MS medium, were transferred onto MS/2 medium for root forming. The plantlets with established root system were transferred to soil and grown under normal greenhouse conditions.

Southern Blot Analysis.

DNA was extracted from the homogenized 2 g leaf tissue by extraction buffer (100 mM Tris-HCl pH 8.0, 50 mM EDTA, 100 mM NaCl, 2% (w/v) SDS and 50 μg/ml proteinase K)²¹. DNA (12 μg) was digested with HindIII or EcoR1, run on a 0.8% (w/v) agarose gel and transferred onto nylon membrane (Hybond N+, Amersham). The following DNA fragments were used as ³²P labelled probes: 421 bp fragment of the bar gene, 888 bp fragment of the rh1A gene, 1281 bp fragment of the rh1B gene, in accordance to the gene analyzed. Standard hybridization conditions for Southern analysis were applied²².

Thin Layer Chromatography (TLC).

Rhamnolipids were isolated from plant tissues by consecutive steps of acid precipitation (pH 2.0) and dissolution in aqueous NaHCO₃ solution (pH 8.6)²³. Components of the partially purified rhamnolipids were analysed by TLC. The rhamnolipids were detected by spraying of anthrone reagent¹⁶. As a control, we used the rhamnolipids extracted from Pseudomonas aeruginosa PAO1.

The Preparation of Soil for Experiments with Copper.

The soil content: peat, sand, and garden soil (1:1:1, v/v/v). The soil was autoclaved (1.5 atm/2 hour). Copper was added in the form of CuSO₄×5H2O salt dissolved in deionized water. The added salt amount: 4200 mg kg⁻¹ wet soil, (β=65%), which in case of Arabidopsis equals to the 1067 mg kg⁻¹ wet soil Cu amount. For tobacco, Cu amount was 912 mg kg⁻¹ wet soil. Soil was mixed with salt and potted. The soil mixture without metal was used as a control.

Preparation of Soil for Experiments with Crude Oil.

The soil mixture is analogous to that with Cu. The added petroleum amounts are: 12973 mg kg⁻¹ wet soil (1.3% contamination) and 23784 mg kg⁻¹ wet soil (2.4% contamination). Soil moisture (β): 65%. Oil was added by manual spraying, and thoroughly mixed to homogeneous state, and, after one day, potted. Crude oil, (Russian URALS brand), chemical composition: density (g/cm³)—0.865; V₅₀—6.28; acid number (mg/g)—0.031; S (%)—1.32; petrolatum (%)—2.13; water—0.11; chlorate content (mg/m³)—0.038. The soil mixture without crude oil was used as a control.

Plant Selection and Growth.

The F₂ seeds of transgenic Arabidopsis plants were grown on MS/2 medium with PPT (20 mg L⁻¹) in Petri dishes. Then, each plant with a root and 2-3 leaves was transferred into soil, and, after 3-4 weeks, an established plant was transferred into soil with heavy metal or crude oil (one plant per-pot).

The F₁ seeds of Nicotiana tabacum plants with rh1A construct were grown on MS/2 medium with Km (200 mg L⁻¹). Then the established plants were transferred into soil for testing of copper accumulation (one plant per pot). During experiments with copper and crude oil contamination the plants were watered in every second day and were not fertilised.

Sample Preparation and Cu Determination.

In the experiments with Arabidopsis thaliana, two best Arabidopsis lines for each of the three constructs were tested (three plants per line). In the experiments with Nicotiana tabacum, one best line of tobacco plants with rh1A construct was tested (three plants per line).

For Arabidopsis, the shoots and rhizosphere were sampled after 45 days and dried at 80° C. The shoots and rhizosphere were separated, weighted and placed in the drying cabinet at 80° C. until constant weight was reached. As for tobacco, only leaves were studied. All samples were consecutively treated with deionized water (2-5 ml), concentrated nitric acid (6-18 ml). Then they were placed on a heating block (Digestor 2040) for the period of 1 hr (for soil) or 6 hrs (for plant tissues). After cooling, the mixtures were heated again in 30% H₂O₂ (3-9 ml) on Digestor 2040/1 hour²⁴. After the next cooling, the mixtures were filtered and diluted with deionized water to 25 ml or to 50 ml subject to the prior sample weight. Cu content was determined on Perkin Elmer Atomic Absorption Spectrometer 3300. Prior to sample analysis, a five-point calibration was established. A midlevel calibration verification sample and blanks were analyzed in every 10^{th} sample. At the end of the analysis, the quality control for metal detection was done. Differences between means were determined with the help of ANOVA in Microsoft Excel.

Sample preparation and the analysis of samples for total petroleum hydrocarbons (PHC).

For each of the three constructs, two best Arabidopsis lines were tested: three plants per line. A 0.1:2.5 g plant tissue/soil sample was mixed with sodium sulfate to dry the sample. The mixtures were extracted with 60 ml of methylene chloride with the help of Accelerated Solvent Extractor ASE 200 (EPA Method 3550) 24, then concentrated to appropriate volumes.

Analysis of soil and plant samples for total petroleum hydrocarbons (PHC) was performed by Gas Chromatography with the help of Flame Ionization Detection (GC/FID) on Hewlett Packard HP6890 Series GC System. The GC/FID conditions were: initial column temperature—35° C.; initial hold time—10 minutes; oven temperature program rate—3° C./minute; final oven temperature—320° C.; final hold time—10 minutes; injection port temperature—275° C.; detector temperature—325° C.; column flow rate (hydrogen)—1 ml/minute; aliquot of the sample extract—1 μL.

Prior to sample analysis, a five-point calibration was established (EPA Method 8100)²⁴. A midlevel calibration verification sample and methylene chloride blanks were analyzed in every 10^{th} sample. It had to meet a calibration criteria. Total petroleum hydrocarbons content (defined operationally as the resolved plus unresolved hydrocarbons eluting between the C₇ and C₃₆ n-alkanes) was also quantified by the internal standards method. A statistical comparison of means was determined with the help of ANOVA in Microsoft Excel.

### REFERENCES

1. Malawska, M., and Wilkomirski, B. 2001. An analysis of soil and plants (Taraxacum officinale) contamination with heavy metals and polycyclic aromatic hydrocarbons (PAHs) in the area of the railway junction Ilawa Glowna, Poland. Water, Air, and Soil Pollution 127:339-349.

2. Semple, K. T., Reid, B. J., and Fermor, T. R. 2001. Impact of composting strategies on the treatment of soils contaminated with organic polutants. Environ Pollution 112:269-283.

3. Douglas, G. S., McCarthy, K. J., Dahlen, D. T., Seavey, J. A., Steinhauer, W. G., Prince, R. C., and Elmendorf, D. G. 1992. The use of hydrocarbon analysis for environmental assessment and remediation. J Soil Contamination 1(3):197-216.

4. Cunningham, S. D., Berti, W. R., and Huang, J. W. 1995. Phytoremediation of contaminated soils. Trends in biotechnology 13:393-397.

5. Mejare, M., and Bullow, L. 2001. Metal-binding proteins and peptides in bioremediation and phytoremediation of heavy metals. Trends in Biotechnology 19(2):67-72.

6. Dahmani-Muller, H., van Oort, F., Gelie, B., and Balabane, M. 2000. Strategies of heavy metal uptake by tree plant spesies growing near a metal smelter. Environ Pollution 109:231-238.

7. Swannel, R. J., and Head, I. M. 1994. Bioremediation comes of ages. Nature 368:396-397.

8. Karenlampi, S., Schat, H., Vangronsveld, J., Verkleij, J. A. C., van der Leie, D., Mergeay, M., and Tervahauta, A. I. 2000. Genetic engineering in the improvement of plants for phytoremediation of metal polluted soils. Environ Pollution 107:225-231.

9. Chayabutra, C., and Ju, L.-K. 2000. Degradation of n-hexadecane and its metabolites by Pseudomonas aeruginosa under microaerobic denitrifying conditions. Appl Environ Microbiol 66:493-498.

10. Ochsner, U. A., Fiechter, A., and Reiser, J. 1994. Isolation, characterization, and expression in Escherichia coli of the Pseudomonas aeruginosa rh1AB genes encoding a rhamnosiltransferase involved in rhamnolipid biosurfactant synthesis. J Biol Chem 269:19787-19795.

11. Desai, J. D., and Banat I. M. 1997. Microbial production of surfactants and their commercial potential. Microbiol Mol Biol Rev 61:47-64.

12. Jarvis, F. G., Johnson, M. J. 1949. A glycolipid produced by Pseudomonas aeruginosa. J Am Chem Soc 71:4124-4126.

13. Ochsner, U. A., Koch, A. K., Fiechter, A., and Reiser, J. 1994. Isolation and characterization of a regulatory gene affecting rhamnolipid biosurfactant synthesis in Pseudomonas aeruginosa. J Bacteriol 176: 2044-2054.

14. Mulligan, C. N., Yong, R. N., and Gibbs, B. F. 2001. Surfactant-enchanced remediation of contaminated soil: a review. Engin Geol 60:371-380.

15. Torrens, J. L., Hermann, D. C., and Miller-Maier, R. M. 1998. Biosurfactant (rhamnolipid) sorption and the impact on the rhamnolipid-facilitated removal of cadmium from various soils under saturated flow conditions. Environ Sci Technol 32(6):776-781.

16. Wild, M., Caro, A. D., Miller, R. M., and Soberon-Chavez, G. 1997. Selection and partial characterization of a Pseudomonas aeruginosa mono-rhamnolipid deficient mutant. FEMS Microbiol Lett 153:279-285.

17. Kopponen, P., Utriainen, M., Lukkari, K., Suntionen, S, Karenlampi, M., and Karenlampi, S. 2001. Clonal differences in copper and zinc tolerance of birch in metal-supplemented soils. Environ Pollution 112:89-97.
18. Pezeshki, S. R., Hester, M. W., Lin, Q., and Nyman, J. A. 2000. The effect of oil spill and clean-up on dominant US Gulf coast marsh macrophytes: a review. Environ Pollution 108:129-139.

19. Editorial article. 1997. Nature Biotechnology 15(5):37.

20. Potrycus, I., Miffelsten Scheid, O., Sauffer, C., Spangenberg, G. 1991. Gene transfer to plants. EMBO advanced laboratory course: 124.
21. McCouch, S. R., Kochet, G., Yu, Z. H., Wang, Z. Y., Khush, G. S., Coffman, W. R., Tanksley, S. D. 1988. Molecular mapping of rice chromosomes. Theor Appl Genet 76:815-829.

22. Southern, E. M. 1975. Detection of specific sequences among DNA fragments separated by gel electrophoresis. J Mol Biol 98:503-517.

23. Zhang, Y., and Miller, R. M. 1992. Enchancement of octadecane dispersion and biodegradation by a Pseudomonas rhamnolipid surfactant (biosurfactant). Appl Environ Microbiol 58:3276-3282.

24. US EPA 1986. Test methods for evaluating solid waste: physical/chemical methods, 3^{rd} edn. SW+846 9060 U.S. Environmental Protection Agency office of solid waste and emergency response. Washington, DC.

1. A method of phytoremediating an environment which is contaminated with at least one heavy metal or oil hydrocarbon, which method comprises:
(a) providing a transgenic plant, which plant expresses at least one heterologous nucleic acid encoding an enzyme having rhamnosyltransferase activity,
(b) planting or locating said transgenic plant in said environment.

2. A method as claimed in claim 1 wherein the environment is contaminated with both heavy metal and oil hydrocarbon pollutants.

3. A method as claimed in claim 1 wherein the plant expresses two different heterologous enzymes having rhamnosyltransferase activity and said environment is optionally contaminated with both heavy metal and oil hydrocarbon pollutants.

4. A method of producing a transgenic plant having improved phytoremediating properties with respect to heavy metal or oil hydrocarbon pollutants, the method comprising:
(i) introducing into a plant cell a heterologous nucleic acid vector encoding at least one enzyme having rhamnosyltransferase activity,
(ii) causing or allowing recombination between the nucleic acid vector and the plant cell genome to introduce the nucleotide sequence encoding the enzyme having rhamnosyltransferase activity into the plant genome,
(iii) regenerating the transformed plant cell into a plant, such that the or each enzyme is expressed in the plant.

5. A method as claimed in claim 4 wherein the vector encodes two different enzymes having rhamnosyltransferase activity.

6. A method as claimed in any one of the claim 3 wherein the phytoremediating is either one or both of phytostabilizing heavy metal pollutants or phytodegrading oil hydrocarbons.

7. A method as claimed in claim 6 wherein the heavy metal is selected from the group consisting of at least one of lead, copper, cadmium, nickel, mercury, arsenic, selenium, strontium or zinc.

8. A method as claimed in any one of the claim 3 wherein the oil hydrocarbon is crude oil.

9. A method as claimed in claim 3 wherein the oil hydrocarbon is the C₁₂-C₁₈ hydrocarbon fraction of crude oil.

10. A method as claimed in claim 3 wherein the metal accumulation coefficient (C_{MA}) of the plant:
(C_{MA}=[Cₛ]/[Cᵣ], where where the heavy metal is copper present at 1000 mg/kg, and Cₛ is the copper concentration in shoot, and Cᵣ is the copper concentration in the rhizosphere, is less than 20% of that of a corresponding non-transgenic plant.

11. A method as claimed in claim 3 wherein at least one enzyme is involved in the synthesis of monorhamnolipids.

12. A method as claimed in claim 11 wherein at least one enzyme is selected from the group consisting o rh1A gene or rh1B gene.

13. A method as claimed in claim 12 wherein the rh1A and rh1B gene are derived from a procaryote.

14. A method as claimed in claim 13 wherein the rh1A and rh1B gene are derived from Pseudomonas aeruginosa.

15. A method as claimed in claim 3 wherein the plant is selected from the group consisting of Nicotiana tabacum; Arabidopsis thaliana.

16. A recombinant plant vector which comprises a nucleotide sequence encoding at least one enzyme having rhamnosyltransferase activity.

17. A vector as claimed in claim 16 wherein the vector encodes two different enzymes having rhamnosyltransferase activity.

18. A vector as claimed in claim 16 wherein at least one enzyme is selected from the group consisting of: rh1A gene or rh1B gene.

19. A vector as claimed in claim 18 wherein the rh1A and rh1B genes are derived from a procaryote.

20. A vector as claimed in claim 19 wherein the rh1A and rh1B genes are derived from Pseudomonas aeruginosa.

21. A plant host cell containing or transformed with a heterologous vector of claim 16.

22. A transgenic plant transformed with a erologous vector of claims 16, or which is a clone, or selfed or hybrid progeny or other descendant of said transgenic plant, which in each case expresses at least one heterologous nucleic acid encoding an enzyme having rhamnosyltransferase activity.

23. A plant as claimed in claim 22, wherein the plant is selected from the group consisting of Nicotiana tabacum and Arabidopsis thaliana.

24. (canceled)

25. A method as claimed in claim 5 wherein the phytoremediating is either one or both of phytostabilizing heavy metal pollutants or phytodegrading oil hydrocarbons.

26. A method as claimed in claim 5 wherein the heavy metal is selected from the group consisting of at least one of lead, copper, cadmium, nickel, mercury, arsenic, selenium, strontium and zinc.

27. A method as claimed in claim 5 wherein the oil hydrocarbon is crude oil.

28. A method as claimed in claim 5 wherein the oil hydrocarbon is the C₁₂-C₁₈ hydrocarbon fraction of crude oil.

29. A method as claimed in claim 5 wherein the metal accumulation coefficient (C_{MA}) of the plant:
(C_{MA}=[Cₛ]/[Cᵣ], where the heavy metal is copper present at 1000 mg/kg, and Cₛ is the copper concentration in shoot, and Cᵣ is the copper concentration in the rhizosphere, is less than 20% of that of a corresponding non-transgenic plant.

30. A method as claimed claim 5 wherein at least one enzyme is involved in the synthesis of monorhamnolipids.

31. A method of phytoremediating an environment which is contaminated with at least one heavy metal or oil hydrocarbon, which method comprises:
(a) providing a transgenic plant, which plant expresses at least one heterologous nucleic acid encoding an enzyme having rhamnosyltransferase activity,
(b) planting or locating said transgenic plant in said environment, wherein said nucleic acid is encoded by the vector of claim 16.

32. A method of producing a transgenic plant having improved phytoremediating properties with respect to heavy metal or oil hydrocarbon pollutants, the method comprising:
(i) introducing into a plant cell a heterologous nucleic acid vector according to claim 16 encoding at least one enzyme having rhamnosyltransferase activity,
(ii) causing or allowing recombination between the nucleic acid vector and the plant cell genome to introduce the nucleotide sequence encoding the enzyme having rhamnosyltransferase activity into the plant genome,
(iii) regenerating the transformed plant cell into a plant, such that the or each enzyme is expressed in the plant.


SEQUENCE LISTING (2 sequences)

SEQ ID NO: 1 - DNA, 33 bp, Artificial Sequence
  primer
tttatcgattgggaggtgtgaaatgcggcgcga

SEQ ID NO: 2 - DNA, 33 bp, Artificial Sequence
  primer
ttttctagatgttcaggcgtagccgatggccat